(19) United States
(12) Patent Application Publication
(10) Pub. No.: US 2020/0139012 A1
(43) Pub. Date: May 7, 2020

(54) METHODS AND MATERIALS FOR IMMUNOMODULATION OF TISSUE GRAFTS

(71) Applicant: UNIVERSITY OF WYOMING, Laramie, WY (US)

(72) Inventor: Jared BUSHMAN, Laramie, WY (US)

(21) Appl. No.: 16/676,077

(22) Filed: Nov. 6, 2019

Related U.S. Application Data
(60) Provisional application No. 62/756,424, filed on Nov. 6, 2018.

(51) Int. Cl. A61L 27/36; A61K 31/573; A61K 47/10; A61L 27/48; A61L 27/54

(52) U.S. Cl. CPC A61L 27/3675; A61K 31/573; A61L 2430/32; A61L 27/48; A61L 27/54; A61K 47/10

(57) ABSTRACT

Embodiments described herein relate to restorative solutions for segmental peripheral nerve (PN) defects using allografted PNs for stimulating PN repair. More specifically, embodiments described herein provide for localized immunosuppression (LIS) surrounding PN allografts as an alternative to systemically suppressing a patient's entire immune system. Methods described herein provide for injection of ISV agents into a nerve graft prior to implantation of the nerve graft into a recipient. Methods described herein also provide for injection of ISV agents with a polymerizable carrier into a nerve graft, polymerization of the carrier within the graft, and implantation of the nerve graft into a recipient. Certain embodiments provide for reinnervation of central nervous system (CNS) axons in a spinal cord utilizing a peripheral nerve graft.

### CROSS-REFERENCE TO RELATED APPLICATIONS

This application claims priority to U.S. Provisional Patent Application No. 62/756,424, filed Nov. 6, 2018, the entirety of which is herein incorporated by reference.

### BACKGROUND

### Field

Embodiments of the present disclosure generally relate to methods and materials for peripheral nerve (PN) repair.

### Description of the Related Art

Peripheral nerves (PNs) branch extensively through the body and are fundamental for motor control, sensation, and function of organ systems. Conventional treatment for segmental PN defects include direct coaptation of nerve stumps, insertion of a mixed (motor+sensory) PN autograft, autografted sensory nerves, decellularized/processed allografted nerves, and biodegradable conduits.

Conduits and processed (decellularized) allografts are not currently capable of matching the regeneration associated with sensory autografts (typically the sural nerve), which is typically considered the clinical standard for PN regeneration. PN autografts require a secondary surgery with significant associated morbidity. Moreover, PN autografts suffer from a limited supply of correctly sized nerves. Size mismatch leads to formation of neuromas and poor functional regeneration.

Mixed PN autografts stimulate superior nerve regeneration when compared to sensory autografts, however, mixed nerves are not viable autograft options in most clinical cases. Schwann cells that myelinate motor or sensory axons intrinsically differ with one another, as Schwann cells that associate with motor axons differentially express several biomolecules that facilitate axon growth and guiding motor axons to correctly reinnervate motor branch points. As a result, sensory grafts that lack motor-associated Schwann cells are not an optimal solution for repair of mixed PNs. An additional limitation of autografts and bioengineered strategies is that neither option is ideally suited to bridge segmental nerve defects of complex nerve structures, such as defects that encompass branch points.

PN allografts are contemplated as a solution to the limitations of PN autografts. While harvesting and screening of donor tissue has advanced considerably, continual systemic immunosuppression (SIS) carries substantial risks of opportunistic infections, renal damage, and post-transplant lymphoproliferative disorders. Patient compliance can also be problematic as many patients fail to adhere to their immunosuppressive treatment regimens, resulting in graft failure. These risks outweigh the potential benefit of allografted PNs.

Spinal cord injuries to the central nervous system (CNS) affect thousands of individuals each year. The spinal cord typically does not appreciably regenerate after a spinal cord injury. Thus, spinal cord injuries often leave the individuals who suffer them permanently disabled. Methods and materials for promoting spinal cord regeneration would be a fundamental achievement in advancing spinal cord injuries, however, current methods and materials are often lacking in their efficacy.

Thus, what is needed in the art are improved methods and materials for nerve repair.

### SUMMARY

In one embodiment, a method of nerve graft preparation is provided. The method includes injecting immunosuppressive agents into a nerve graft in a first region of the nerve graft adjacent to a proximal end of the nerve graft and a second region of the nerve graft adjacent to a distal end of the nerve graft.

In another embodiment, a nerve graft material is provided. The material includes a nerve graft which includes immunosuppressive agents disposed within the nerve graft below or within the epineurium of the nerve graft and the immunosuppressive agents are disposed within the nerve graft adjacent to a proximal and a distal end of the nerve graft.

In yet another embodiment, a method of nerve grafting is provided. The method includes harvesting a nerve graft from a donor, injecting immunosuppressive agents into proximal and distal regions of the nerve graft; and implanting the nerve graft into a host.

### BRIEF DESCRIPTION OF THE DRAWINGS

The patent or application file contains at least one drawing executed in color. Copies of this patent or patent application publication with color drawings) will be provided by the Office upon request and payment of the necessary fee.

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1A illustrates data regarding the toxicity of cyclosporine A (CsA) to primary dorsal root ganglion sensory neurons (DRGs), motor neurons, and Schwann cells according to embodiments described herein.

FIG. 1B illustrates data regarding the toxicity of tacrolimus to DRGs, motor neurons, and Schwann cells according to embodiments described herein.

FIG. 1C illustrates data regarding the toxicity of prednisolone to DRGs, motor neurons, and Schwann cells according to embodiments described herein.

FIG. 2A illustrates data regarding the viability of DRGs, motor neurons, and Schwann cells cultured in about 1 μM CsA with differing concentrations of prednisolone according to embodiments described herein.

FIG. 2B illustrates data regarding the viability of DRGs, motor neurons, and Schwann cells cultured in about 10 μM tracrolimus with differing concentrations of prednisolone according to embodiments described herein.

FIG. 3A illustrates a micrograph of a β-III tubulin stained allograft with no immunosuppression according to embodiments described herein.

FIG. 3B illustrates a micrograph of a β-III tubulin stained allograft with localized immunosuppression according to embodiments described herein.

FIG. 3C illustrates a micrograph of a β-III tubulin stained allograft vehicle according to embodiments described herein.

FIG. 4A illustrates compound muscle action potential data of the peroneal branch of the sciatic nerve after PN allograft repair according to embodiments described herein.

FIG. 4B illustrates compound muscle action potential data of the tibial branch of the sciatic nerve after PN allograft repair according to embodiments described herein.

FIG. 5A illustrates a therapeutic delivery vehicle according to embodiments described herein.

FIG. 5B illustrates a therapeutic delivery vehicle according to embodiments described herein.

FIG. 5C illustrates a therapeutic delivery vehicle according to embodiments described herein.

FIG. 5D illustrates a hydrogel system according to embodiments described herein.

FIG. 6 illustrates a perspective sectional view of a nerve graft injected with immunosuppressive agents according to embodiments described herein.

FIG. 7A illustrates a schematic view of a peripheral nerve graft with immunosuppressive agents disposed therein according to embodiments described herein.

FIG. 7B illustrates a schematic view of a peripheral nerve loaded with immunosuppressive agents grafted into a host nerve according to embodiments described herein.

FIG. 8A illustrates operations of a nerve graft immunosuppressive agent injection process according to embodiments described herein.

FIG. 8B illustrates operations of a nerve graft immunosuppressive agent injection process according to embodiments described herein.

FIG. 9 illustrates images of a nerve graft injected with different volumes of fluorescent beads and a biomaterial carrier according to embodiments described herein.

FIG. 10A illustrates an image of a nerve graft injected with fluorescent beads within a biomaterial carrier one day after injection according to embodiments described herein.

FIG. 10B illustrates an image of a nerve graft injected with fluorescent beads within a biomaterial carrier seven days after injection according to embodiments described herein.

FIG. 10C is a graph illustrating injection site fluorescent intensity around the injection site for a period of seven days corresponding to the images of FIGS. 10A and 10B according to embodiments described herein.

FIG. 10D is an image illustrating a cross section nerve morphometry at the injection site of FIGS. 10A and 10B from toluidine blue staining according to embodiments described herein.

FIG. 10E illustrates a close up view of the injection site of the nerve graft illustrated in FIG. 10D according to embodiments described herein.

FIG. 11A illustrates a schematic representation of a spinal cord with a spinal cord injury site according to embodiments described herein.

FIG. 11B illustrates a schematic representation of a peripheral nerve graft into the spinal cord injury site of FIG. 11A according to embodiments described herein.

FIG. 11C illustrates a schematic representation of a peripheral nerve graft into the spinal cord injury site of FIG. 11B and reintegration of central nervous system axons within the spinal cord across the injury site according to embodiments described herein.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Embodiments described herein relate to restorative solutions for segmental peripheral nerve (PN) defects using allografted PNs for stimulating PN repair. More specifically, embodiments described herein provide for localized immunosuppression (LIS) surrounding PN allografts as an alternative to systemically suppressing a patient's entire immune system. Methods include localized release of immunosuppressive (ISV) agents are contemplated in one embodiment. Methods also include localized application of immunosuppressive (ISV) regulatory T-cells (Tregs) in other embodiments. Hydrogel carrier materials for delivery of ISV agents and are also described herein.

Localized immunosuppression is defined herein as delivering immunosuppressive agents in the local environment of a peripheral nerve allograft, thereby eliminating or minimizing the utilization of immunosuppressive agents for systemic delivery following implantation of a peripheral nerve allograft to treat a segmental peripheral nerve defect. Immunosuppressive agents are defined herein as therapeutics with biological activity that suppresses the activation and activity of the immune response against allografted peripheral nerve tissue. Immunosuppressive agents include, but are not limited to, small molecule therapeutics, peptides, proteins, glycans, antibodies and cells. Cells which may suppress an immune response to grafted tissue include, but are not limited to, T cells (Tregs), mesenchymal stromal cells (MSCs), and adipose-derived stem cells (ADSCs), among others. Such cells may be allogeneic, autologous, or xenogeneic to the recipient.

Methods for delivery of said immunosuppressive agents, encompassing all methods for localized delivery of said immunosuppressive agents to the allograft site, include, but are not limited to, microfluidics and catheterization to deliver agents from internal or external devices to the allograft site, implantation of biocompatible biomaterial carriers in local proximity to the allorograft, such as, biomaterials including poly(ethylene glycol), poly(lactic acid), poly(lactic-co-glycolic) acid, collagen, and fibrin, among others, that release said agents to the allograft, and other pharmaceutical formulations for localized release.

Allografts are contemplated as a solution to the limitations of PN autografts. Within an allografted PN there are the same support cells and structure as are found in autografted PNs, but allografted nerves can be mixed. Allografted mixed PNs stimulate regeneration of injured PNs to the same extent or superior to mixed autologous nerves. Allografts additionally have the potential to bridge complex segmental nerve defects, such as branch points, as PN allografts may be harvested from a donor to the exact region corresponding to the segmental defect in the recipient. Such nerves can be correctly sized for length and diameter to avoid neuromas.

Allografted PNs are distinct relative to other tissues with respect to immunosuppression (ISN). The degree of recovery promoted by allografted PNs is believed to be equally effective when systemic immunosuppression (SIS) is stopped after the axons have regenerated when compared to continuous SIS.

Schwann cells are both the major antigenic components of allografted PNs and the cell type providing much of the regenerative stimulus. It is contemplated that once the motor and sensory axons cross through the allografted nerve segment to innervate distal targets, the allogeneic Schwann cells either lower their immunogenic profile upon myelination of host axons or that the allogeneic Schwann cells can be targeted by the immune system without long lasting effects on the regenerated nerve.

It is contemplated that by delaying the immune response to the graft, a sufficient delay in the immune response to a PN allograft can be achieved through local application of immunosuppressants instead of systemic application of immunosuppressants. Localized immunosuppression minimizes the risks associated with ISV therapy. For example, local administration of ISV agents leaves the immune response in the rest of the body largely intact. In addition, lower doses of ISV agents administered locally are much less likely to cause kidney damage as comparatively negligible amounts of ISV agents enter the circulatory system.

Still further, localized and temporary immunosuppression is much less likely to contribute to causing lymphoproliferative disorders. Moreover, patient compliance is not a concern if sufficient administration of immunosuppressive agents for the entire recovery period can be applied during initial surgery. It is believed that allografted PNs may improve functional recovery of segmental PN defects because allografted PNs take advantage of temporary immunosuppression, thereby facilitating clinical adoption for PN repair.

It is also contemplated that localized immunosuppression through localized delivery of immunosuppressive agents will remove or minimize utilization of systemic immunosuppression. For example, instead of daily systemic immunosuppression for peripheral nerve allografts during the initial regeneration period, systemic immunosuppression may be delivered on a less frequent basis, thus, improving the quality of care for the patient and improving prospects for patient compliance.

Acute rejection of nerve transplants is primarily mediated by T-cells. Following transplantation of a nerve segment, antigen presenting cells (APCs) of donor or host origin activate host T-cells by displaying alloantigens via major-histocompatibility complex (MHC) molecules to the αβT-cell receptors (TCRs) of T-cells, with CD4+ and CD8+ T-cells the primary effector T-cells. Activation of T-cells by APCs can occur locally to the site of the allograft, however, activation of T-cells is more robust when the APCs migrate to a lymphatic center. Chronic rejection occurring over months to years involves B-cell activation and progressive infiltration of macrophages into the graft vasculature. As PN allografts utilize the benefits of temporary immunosuppression, which may span weeks to months, it is contemplated that PN allografts are not subject to functional impairment associated with the timeline of chronic rejection. Accordingly, embodiments described herein provide for a reduced response of effector T-cells to enable PN allograft acceptance via localized immunosuppression.

Calcineurin inhibitors (CNIs), such as cyclosporine A (CsA) and tacrolimus (examples of immunosuppressive agents), are contemplated for utilization with PN allograft transplantation. CNIs prevent activation of T-cells by binding to immunophilins and increasing their affinity for calcineurin, thereby inhibiting calcineurin activation of NFAT and NFkB transcription. CNIs are also associated with preventing or reducing production of IL-2 and other immune activating cytokines. Interestingly, both tacrolimus and CsA have neuroprotective effects even in the absence of transplanted cells or tissues.

Prednisolone is the active form of the glucocorticoid prednisone, a pro-drug metabolized into prednisolone, which may be utilized as another example of an immunosuppressive agent and may be utilized for PN allografts according to embodiments described herein. Prednisolone may also be utilized in combination with CNIs for PN allografts in other embodiments. Anti T-cell antibodies, mammalian or otherwise, are also contemplated for localized immunosuppression to protect against the eventuality that APCs will migrate to lymphatic centers and activate effector T-cells outside of the zone of localized immunosuppression. Suitable anti T-cell antibodies for utilization in human subjects include, but are not limited to ATGAM, visilizumab, alemtuzumab, basiliximab, and daclizumab, among others.

Binding of anti T-cell antibodies (such as R73 and MRC Ox-8, rat model described infra) to αβTCRs on CD4+ and CD8+ cells mitigates the risk of APC migration. Moreover, anti T-cell antibodies may substantially prevent or reduce TCR-MHC binding and target T-cells for depletion via opsonization for phagocytosis or lysis due to complement activation against the T-cell. Tacrolimus, prednisolone, and anti T-cell antibodies referred to herein are also contemplated within the definition of immunosuppressive agents.

Tregs are a sub-population of CD4+ cells and suppress activated effector T-cells through a variety of mechanisms linked to Treg FoxP3 expression. Generally, Tregs function of APCs and effector T-cell populations and proliferate in response to IL-2 and down-regulate the adaptive immune response of effector T-cells. Tregs also attenuate graft versus host disease and a number of other autoimmune disorders. Accordingly, embodiments described herein contemplate local application of Tregs for suppression of an acquired immune response to PN allografted tissue. In addition, mesenchymal stromal cells (MSCs) are also an immunosuppressive cell type contemplated for localized delivery suppress the immune response to allografted PNs as MSCs attenuate many autoimmune diseases and deactivate effector T-cells.

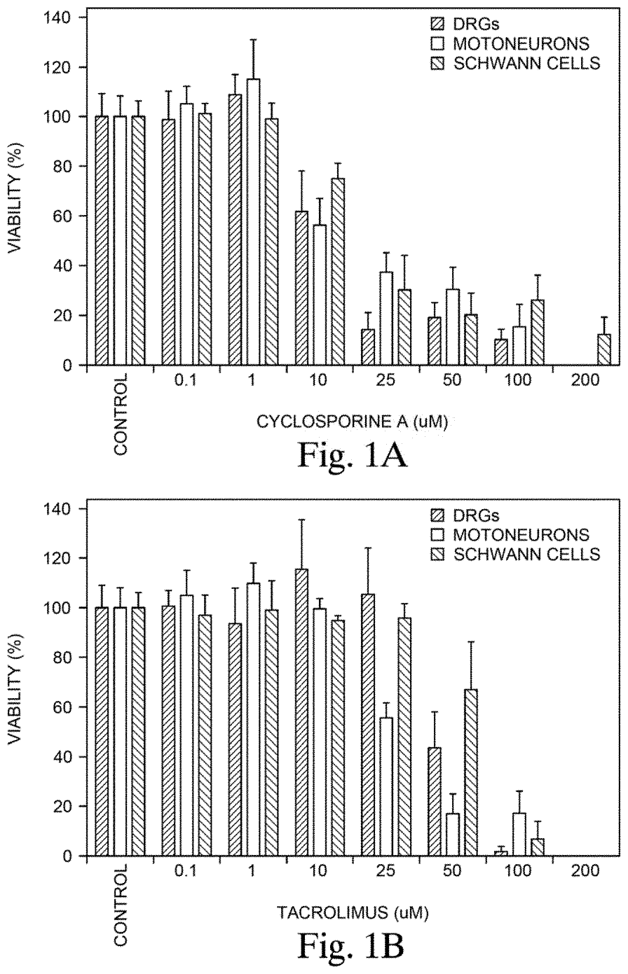

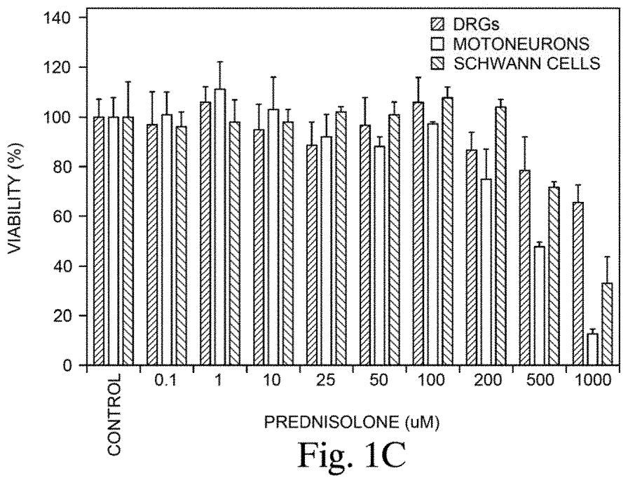

It is contemplated that delivery of immunosuppressive agents function within a favorable therapeutic window. PNs are comprised of three major nervous system cell types, motor neurons, sensory neurons, and Schwann cells. FIG. 1A illustrates data regarding the toxicity of CsA to dorsal root ganglion sensory neurons (DRGs), motor neurons, and Schwann cells. FIG. 1B illustrates data regarding the toxicity of tacrolimus to DRGs, motor neurons, and Schwann cells. FIG. 1C illustrates data regarding the toxicity of prednisolone to DRGs, motor neurons, and Schwann cells.

Cells (DRGs, motor neurons, and Schwann cells) were cultured for 72 hours in various concentration of the immunosuppressive drugs and viability was evaluated using the Alamar blue viability assay. ISV's were diluted into media using a maximum of 1% dimethylsulfoxide (DMSO). The results indicated in FIG. 1A indicate that CsA is tolerated by the cells at doses up to about 1 μM, which is significantly greater than the effective dose of CsA (about 25 μM) for inhibiting activation and proliferation of the immune cells.

The results indicated in FIG. 1B indicate that tacrolimus is tolerated by the cells at doses up to between about 10 μM and about 25 μM, which is greater than the effective dose of tacrolimus (between about 50-100 μM) for inhibiting activation and proliferation of immune cells. The results indicated in FIG. 1C indicate that prednisolone is tolerated by the cells at doses up to about 200 μM, which is greater than the effective dose of prednisolone (greater than about 240 μM) for inhibiting activation and proliferation of immune cells

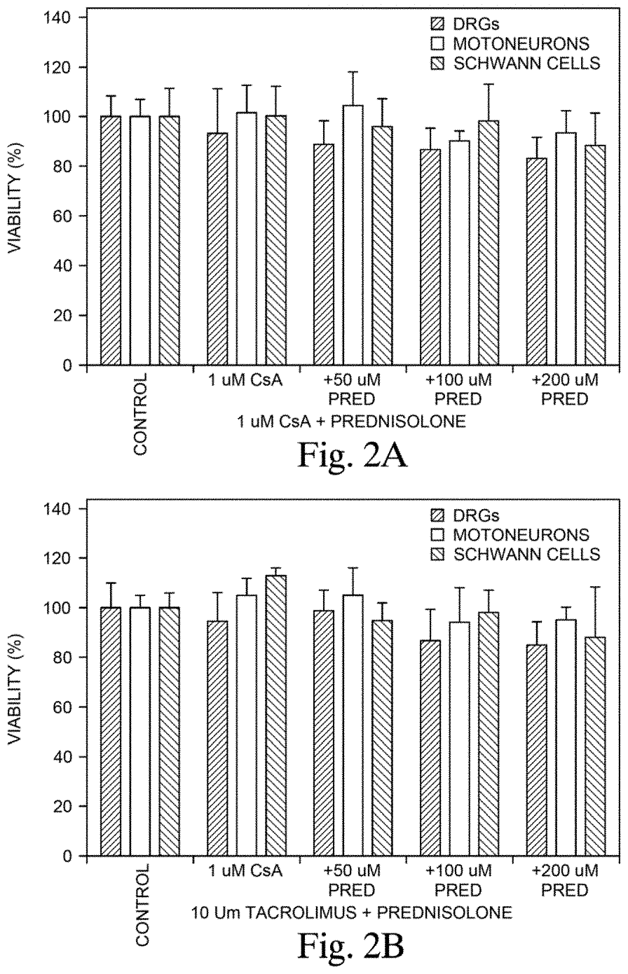

FIG. 2A illustrates data regarding the viability of DRGs, motor neurons, and Schwann cells cultured in about 1 μM CsA with differing concentrations of prednisolone. FIG. 2B illustrates data regarding the viability of DRGs, motor neurons, and Schwann cells cultured in about 10 μM tracrolimus with differing concentrations of prednisolone. In other words, the cells were exposed to the highest tolerated doses of CsA and tacrolimus with increasing doses of prednisolone and no toxicity was observed. Thus, it is contemplated that CNIs may be utilized in combinations with prednisolone for localized immunosuppression.

### Experimental Methods

Biological material from Sprague Dawley (GFP− and GFP+) and Lewis rats, without any pathological conditions, were utilized to determine the PN allograft methods and materials described herein. Lewis rats were obtained from Charles River Laboratories (Wilmington, Mass.) and Sprague Dawley rats were obtained from Rat Resource and Research Center (Columbia, Mo.). Alzet osmotic pumps (model 1004), commercially available from DURECT Corporation, Cupertino, Calif., were utilized for delivery of immunosuppressive agents to PN allografts.

For animal experiments, Sprague Dawley (SD) PN allografts were implanted within Lewis recipients, which are respectively RT1¹ and RT1^{b} for major histocompatibility complex (MHC). Donor sciatic nerves are harvested from SD rats under isofluorane anesthesia and connective and other non-nerve tissue was removed. Under sterile “no touch” techniques, the left sciatic nerve of recipient Lewis rats was exposed and a segment of the host nerve was removed that corresponds to a length of the allograft being inserted, spanning approximately 2 mm distal for the length of the allografted segment to be inserted. 9-0 sutures are used to insert the SD allograft PN.

After suturing the allograft PN into place, osmotic pumps were implanted subcutaneously approximately 2 cm lateral to the surgical cavity. A polyethylene catheter extends from the pump into the tissue cavity, where it is sutured to the suture line that closes the musculature, with approximately 0.75 cm of the catheter remaining within the cavity to release ISV agents. The osmotic pump released 0.11 μl/hr of solution for 28-38 days. The positive control group of daily systemic immunosuppression received intraperotineal (IP) injections of 1 mg/kg tacrolimus the day before surgery and every day thereafter. LIS and vehicle control (70% dimethylsulfoxide (DMSO) 30% phosphate buffered saline (PBS)) received IP injections of tacrolimus the day before and the day of surgery.

To extrapolate the in vitro effective and tolerated doses of ISV agents to PN and immune cells to a dynamic in vivo environment, the tissue cavity with the sciatic nerve contained 190±22 μl of fluid after suturing the muscles together. This figure was used for the volume in dosage calculations. Several assumptions were made regarding the flow of interstitial fluid (i.e. turnover within the cavity) in order to calculate drug loading. Based on the osmotic pressure in rat skeletal muscles (−1 to −3 mm Hg) compared to −0.20 mm Hg for the overall body average, brain tissues ranging from +3 to +8 mm Hg and the rate of lymphatic turnover in general, it was postulated that the fluid within the cavity containing the allograft and pump would turnover hourly. On the basis of these measurements, the experiment with parameters outlined in Table 1 was performed.

TABLE 1

| | in vitro | | Measures in | | Effective | Targeted | | Concentrations | |
|---|---|---|---|---|---|---|---|---|---|
| | effective | | vitro tolerated | | clinical | concentration | | loaded with | |
| | concentration | | concentrations | | plasma | within the | | implanted | |
| ISV agent | on immune cells | | on PN cells | | concentration | nerve cavity | | osmotic pump | |
| Tacrolimus | 0.5-1.0 | nM | 10-25 | μM | 21 nM | 5 | μM | 9 | mM |
| Prednisolone | 240 | nM | 200 | μM | 277 nM | 50 | μM | 90 | mM |

| | | | | | | | | |
|---|---|---|---|---|---|---|---|---|
| Anti-TCR | 10 | ng/ml | N/A | N/A | 165 | ng/ml | 30 | μg/ml |
| antibody | | | | | | | | |

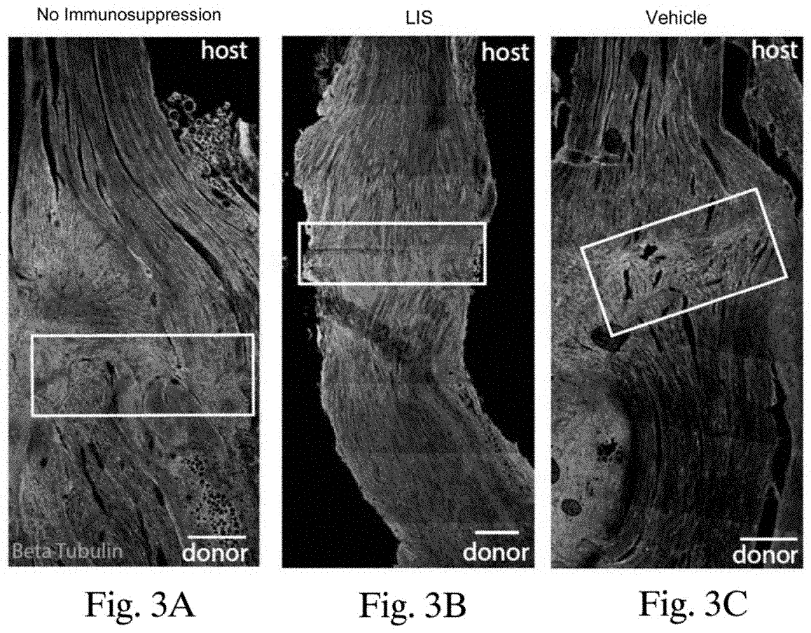

Rats were sacrificed after 1, 4, 6, and 16 weeks following surgery. Longitudinal sections of the nerves were cut and labeled for markers of axons (β-III tubulin—Abcam 1:200) and T-cells (R73—Cederlane Laboratories 1:200). Sections showed that regeneration of axons into the allografts in recipients of LIS was comparable to the positive control of daily SIS. After 1 week, host axons had crossed into the allografts of SIS and LIS recipient animals in line with the organization of the nerve. In contrast, β-III tubulin stains in no ISN and vehicle control animals showed a disorganization of axons and denser accumulation of fibrous tissue at the donor-host boundary as illustrated in FIGS. 3A and 3C.

After 28 days, allografts with LIS showed axons extending in line from the host nerve into and through the allograft as illustrated in FIG. 3B. The proximal and distal host-donor boundaries, as determined by identifying the proximal and distal suture points, were histologically indistinguishable in LIS recipients, indicating a functional merger of tissues.

In contrast, negative control groups of vehicle and no ISN showed a lack of axonal crossing into the allograft and robust accumulation of T-cells and apparent scar tissue at the donor-host boundary as illustrated in FIGS. 3A and 3C. Fluorescent densitometry of β-III tubulin was conducted for 10 randomly selected sections within 1 cm² segments, taking the ratio of the signal within the allograft to the proximal host structure, showing a 78±11% reduction in signal intensity of β-III tubulin in the allograft regions of vehicle controls compared to LIS. Accordingly, it is believed that LIS for 28 days is effective at promoting robust regeneration of host axons into allografted PNs, while the negative controls exhibit graft rejection and lack of host axonal entry into the graft which is indicative of long-term graft failure.

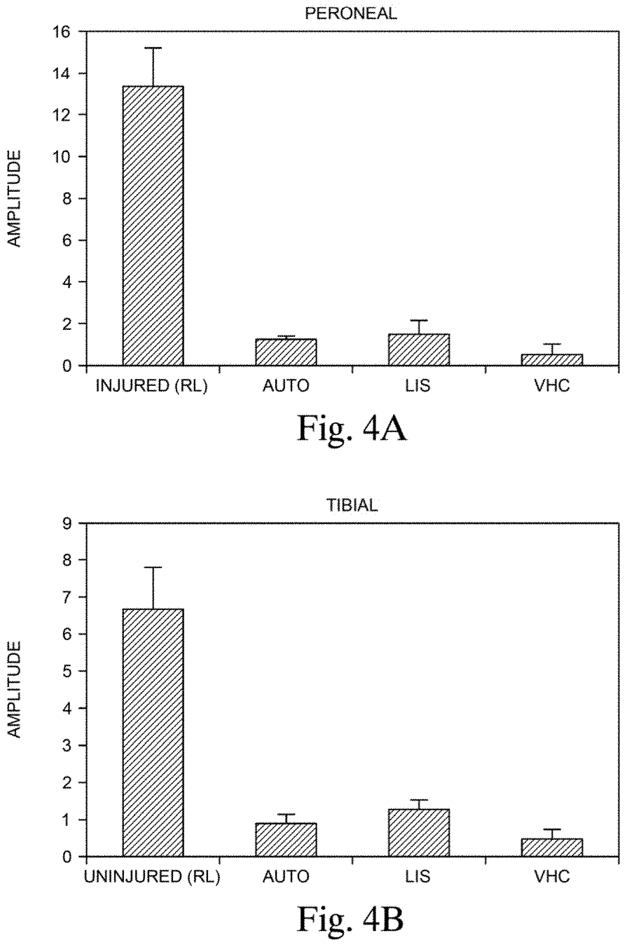

FIG. 4A illustrates compound muscle action potential (CMAP) data of the peroneal branch of the sciatic nerve after PN allograft repair in the rat experiment described above. FIG. 4B illustrates compound muscle action potential data of the tibial branch of the sciatic nerve after PN allograft repair. CMAPs (electrophysiological measurements) were obtained at the dorsal and planta foot muscles, which are the most distal reinnervation targets of the peroneal and tibial branches of the sciatic nerve. CMAP amplitude measures the strength of nerve signal conduction and corresponds to the number of conducting axons of the regenerated nerve that reached the muscles.

FIG. 4A data, which is based on CMAP tests of the peroneal branch of the sciatic nerve 16 weeks post PN allograft, indicates that PN allografts utilizing localized immunosuppression were at least equivalent to autologous grafts and better than the vehicle. FIG. 4B data, which is based on CMAP tests of the tibial branch of the sciatic nerve 16 weeks post PN allograft, indicates that PN allografts utilizing localized immunosuppression were at least equivalent to autologous grafts and better than the vehicle. Accordingly, it can be seen that localized immunosuppression utilized with a PN allograft provides for desirable PN regeneration without the risks of systemic immunosuppression.

### Materials for ISV Agent Delivery

Embodiments described herein provide for materials for ISV agent delivery in a biological system. Materials described herein are contemplated to enable ISV agent delivery for a period of several days, to several weeks, to several months, for example an amount of time greater than one month, such as 2 months or greater. Further, the materials described herein provide an encapsulant that delivers Tregs and/or MSCs to the PN allograft. The materials are contemplated to enable controlled release profiles of ISV agents, Tregs cells, antibodies, MSCs, and cells, via characteristics of the material composition. A degradation motif, a macromolecular structure, a macromere mass fraction, and a particle size distribution are controllable to enable continual and controlled release of the ISV agents, Tregs cells, antibodies, MSCs, and cells. Moreover, the materials described herein exhibit desirable mechanical flexibility and strain moduli for in vivo application, biodegradability, and bioresorbability, thus, providing additional benefits for a wide range of nerve repair application with reduced morbidity risks.

Microfluidic co-emulsification of biomaterials, ISV, Tregs cells, antibodies, MSCs, and cells enables fabrication of layered particles and confers versatility for application in PN allograft systems with localized immunosuppression. In one embodiment, hydrophobic small molecule therapeutics are formulated into surface-eroding, poly(lactic-co-glycolic acid) (PLGA) polymeric nanoparticles, which are subsequently doped into bulk-eroding material fabricated from poly(ethylene glycol) (PEG) diacrylate (PEG-DA) or poly(ethylene glycol) norbornene (PEG-NB). Thus, nanoparticles are dispersed into a bulk hydrogen material. In one embodiment, the surface-eroding nanoparticles are fabricated from a hydrogel material. In another embodiment, the bulk-eroding material is fabricated from a hydrogel material. In one embodiment, the surface-eroding nanoparticles and the bulk-eroding material are fabricated from the same hydrogel material. Alternatively, the surface-eroding nanoparticles and the bulk-eroding material are different hydrogel materials.

In one embodiment, the hydrogel utilized for the bulk-eroding material is a photopolymerizable hydrogel, however, it is contemplated that other polymerization techniques may be utilized in accordance with the embodiments described herein. For example, the hydrogel material may be a thermally polymerizable hydrogel and/or a chemically polymerizable hydrogel. In another embodiment, the hydrogel utilized for the polymeric nanoparticles is a photopolymerizable hydrogel. The disclosure of Methods of Generating Microparticles and Porous Hydrogels Using Microfluidics in U.S. patent application Ser. No. 15/335,184 is hereby incorporated by reference in its entirety.

Hydrogel materials, which are pharmaceutically acceptable carriers for the various ISV agents, Tregs cells, antibodies, MSCs, and cells described above, are those materials which are considered to be substantially compatible (i.e. non-toxic or a biologically acceptable degree of toxicity) with biological tissue. In one embodiment, the hydrogel materials are hydrophilic. In another embodiment, the hydrogel materials include three-dimensional polymeric networks having polymer chains cross-linked by either covalent bonds or physical interactions, such as entanglements and/or crystallites. In one embodiment, the hydrogel materials exhibit characteristics (i.e. hydrophilicity) that enable the hydrogel materials to absorb large amounts of water or biological fluids, thus enhancing biocompatibility. In another embodiment, the hydrogel materials are degradable such that the structural components of the hydrogel materials break down over time and are capable of being absorbed by the biological system into which the hydrogel materials are incorporated. Alternatively, the hydrogel materials are non-degradable.

### Natural Hydrogels

In one embodiment, the hydrogel materials are natural hydrogels which are derived from naturally occurring biomolecules synthesized by living systems. Examples of natural hydrogel materials include, but are not limited to: extracellular matrix (ECM) and chemically modified derivatives thereof; collagen and chemically modified derivatives thereof; alginate and chemically modified derivatives thereof; agarose and chemically modified derivatives thereof; peptide-based hydrogels and chemically modified derivatives thereof; hyaluronic acid and chemically modified derivatives thereof; hyaluronan and chemically modified derivatives thereof; keratin and chemically modified derivatives thereof; fibronectin and chemically modified derivatives thereof; dermatan and chemically modified derivatives thereof; poly(sialic acid) and chemically modified derivatives thereof; fibrin and chemically modified derivatives thereof; chitosan and chemically modified derivatives thereof; chitin and chemically modified derivatives thereof; cholic acid and chemically modified derivatives thereof; dextran and chemically modified derivatives thereof; dextrin and chemically modified derivatives thereof; cellulose and chemically modified derivatives thereof; gelatin and chemically modified derivatives thereof; gelatinous protein mixtures and chemically modified derivatives thereof, for example, MATRIGEL® available from Corning Life Sciences; laminin and chemically modified derivatives thereof; perlecan and chemically modified derivatives thereof; aggrecan and chemically modified derivatives thereof; glycosaminoglycans and chemically modified derivatives thereof; heparin and chemically modified derivatives thereof; cholesterol and chemically modified derivatives thereof; chondroitin sulfate and chemically modified derivatives thereof; polycholesterol and chemically modified derivatives thereof; hydroxyapatite and chemically modified derivatives thereof; silk and chemically modified derivatives thereof; bisphosphonates and chemically modified derivatives thereof; tricalcium phosphate and chemically modified derivatives thereof; sacran and chemically modified derivatives thereof; decellularized extracellular matrix and chemically modified derivatives thereof; xanthum gum and chemically modified derivatives thereof; starch and chemically modified derivatives thereof; pectin and chemically modified derivatives thereof; amylopectin and chemically modified derivatives thereof; aymlose and chemically modified derivatives thereof; and elastin and chemically modified derivatives thereof.

### Synthetic Hydrogels

In one embodiment, the hydrogel materials are synthetic hydrogels which are not typically synthesized by living systems in nature. Examples of synthetic hydrogel materials include, but are not limited to: poly(ethylene glycol) and chemically modified derivatives thereof; poly(glutamic acid) and chemically modified derivatives thereof; poly(propylene fumarate) and chemically modified derivatives thereof; poly(N-hydroxyethyl)-DL-aspartamide and chemically modified derivatives thereof; propylene glycol and chemically modified derivatives thereof; poly(ethylene oxide) and chemically modified derivatives thereof; poly(propylene oxide) and chemically modified derivatives thereof; poly(vinyl alcohol) and chemically modified derivatives thereof; poly(acrylic acid) and chemically modified derivatives thereof; poly(hedral oligosilsesquioxane) and chemically modified derivatives thereof; poly(methacrylic acid) and chemically modified derivatives thereof; poly(vinyl-pyrrolidone) and chemically modified derivatives thereof; poly(isopropylacrylamide) and chemically modified derivatives thereof; polyphosphazene and chemically modified derivatives thereof; peptides and chemically modified derivatives thereof; polyaldehyde and chemically modified derivatives thereof; tyrosine-derived polycarbonates and chemically modified derivatives thereof; methacrylate and chemically modified derivatives thereof; polymethacrylate and chemically modified derivatives thereof; N-isopropylacrylamide and chemically modified derivatives thereof; acrylamide and chemically modified derivatives thereof; polyacrylamide and chemically modified derivatives thereof; acrylate and chemically modified derivatives thereof; polyacrylate and chemically modified derivatives thereof; poly(lactic acid) and chemically modified derivatives thereof; poly(glycolic acid) and chemically modified derivatives thereof; poly(lactide-co-glycolide) and chemically modified derivatives thereof; poly(lactic-co-glycolic acid) and chemically modified derivatives thereof; poly(3,4-ethylenedioxythiophene) and chemically modified derivatives thereof; graphene oxide and chemically modified derivatives thereof; poly(graphene oxide) and chemically modified derivatives thereof; polycaprolactone and chemically modified derivatives thereof; sodium dodecyl sulfate and chemically modified derivatives thereof; vinyl phosphonic acid and chemically modified derivatives thereof; poly-dimethylsiloxane and chemically modified derivatives thereof; titanium and chemically modified derivatives thereof; bioactive glass and chemically modified derivatives thereof; carbon nanotubes and chemically modified derivatives thereof; silicone and chemically modified derivatives thereof; silica and chemically modified derivatives thereof; and thiolene materials and chemically modified derivatives thereof.

In another embodiment, the hydrogel materials are formed from mixtures of two or more of the natural hydrogel materials. In another embodiment, the hydrogel materials are formed from mixtures of two or more of the synthetic hydrogel materials. For example, poloxamers include three distinct synthetic materials arranged as triblock copolymers. It is also contemplated that various polymeric materials described herein may be modulated with functional groups to facilitate fabrication of hydrogel materials which exhibit characteristics selected to enhance delivery of the ISV agents, Tregs cells, antibodies, and cells or to enhance the biocompatibility of the hydrogel material with the biological system within which the hydrogel material is utilized.

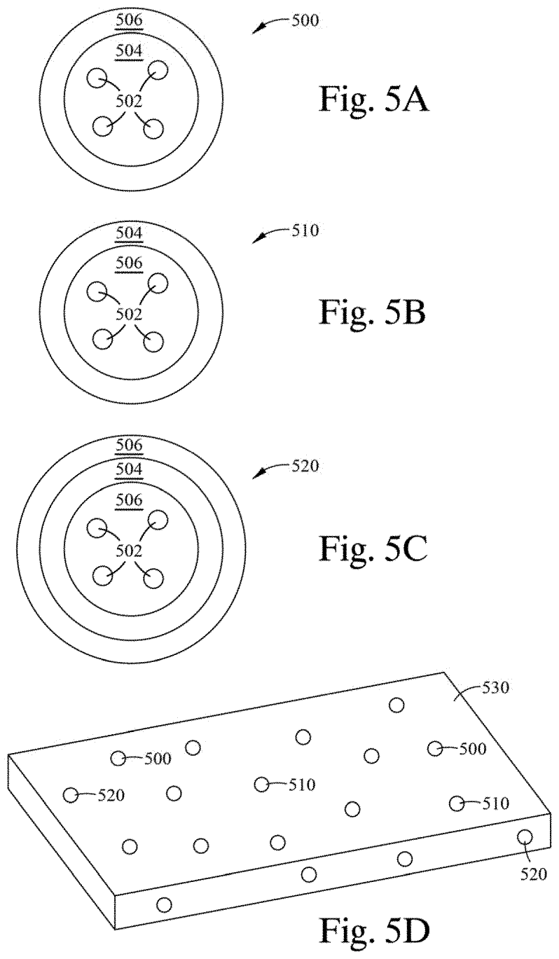

FIG. 5A illustrates a delivery vehicle 500 for ISV agent, Tregs antibody, and/or MSC release. The delivery vehicle 500 is fabricated in a manner to enable time delayed and/or continuous release of the ISV agents and/or Tregs cells, antibodies/MSCs. The delivery vehicle 500 includes a plurality of nanoparticles 502, a first material layer 504, and a second material layer 506. The nanoparticles 502 are interdispersed within the first material layer 504 and the first material layer 504 is encapsulated within the second material layer 506.

In one embodiment, ISV agents, such as CsA, tacrolimus, and/or prednisolone, are dispersed within and supported by the nanoparticles 502. In another embodiment, Tregs cells, antibodies and/or MSCs are dispersed within the nanoparticles 502. In one embodiment, the nanoparticles 502 comprise PLGA, however it is contemplated that other surface eroding materials suitable for utilization in a biological system may be advantageously utilized according to the embodiments described herein.

The first material layer 504 has the nanoparticles 502 disposed therein and in one embodiment, the first material layer 504 comprises an enzymatically degrading PEG material. The second material layer 506 encapsulates the first material layer 504. Either of the first material layer 504 and/or the second material layer 506 are fabricated from one or both of the naturally derived and synthetic hydrogel materials described herein. In one embodiment, the second material layer 506 comprises a hydrolytically degrading PEG material. The enzymatically degrading PEG material and the hydrolytically degrading PEG material have different degradation rates in one embodiment. For example, the hydrolytically degrading PEG material may have a degradation rate greater than a degradation rate of the enzymatically degrading PEG material. In other words, the hydrolytically degrading PEG material decomposes faster than the enzymatically degrading PEG material. In another example, the hydrolytically degrading PEG material may have a degradation rate less than a degradation rate of the enzymatically degrading PEG material. In other words, the hydrolytically degrading PEG material decomposes slower than the enzymatically degrading PEG material. In another embodiment, the enzymatically degrading PEG material and the hydrolytically degrading PEG material have similar degradation rates.

FIG. 5B illustrates a delivery vehicle 510 for ISV agent, Tregs antibody, and/or MSC release. Similar to the delivery vehicle 500, the delivery vehicle 510 is fabricated in a manner to enable time delayed and/or continuous release of the ISV agents and/or Tregs cells, antibodies/MSCs. The delivery vehicle 510 includes the plurality of nanoparticles 502, the first material layer 504, and the second material layer 506. The nanoparticles 502 are interdispersed within the second material layer 506 and the second material layer 506 is encapsulated within the first material layer 504.

FIG. 5C illustrates a delivery vehicle 520 for ISV agent, Tregs antibody, and/or MSC release. Similar to the delivery vehicles 500, 510, the delivery vehicle 520 is fabricated in a manner to enable time delayed and/or continuous release of the ISV agents and/or Tregs cells, antibodies/MSCs. The delivery vehicle 520 includes the plurality of nanoparticles 502, the first material layer 504, and a plurality of second material layers 506. The nanoparticles 502 are interdispersed within the second material layer 506 and the second material layer 506 is encapsulated within the first material layer 504. An additional second material layer 506 encapsulates the first material layer 504. In another embodiment, the nanoparticles 502 may be interdispersed within the first material layer 504 and the first material layer 504 may be encapsulated with the second material layer 506. An additional first material layer 504 may encapsulate the second material layer 506.

FIG. 5D illustrates a bulk hydrogel material 530 having a plurality of delivery vehicles disposed therein. In one embodiment, the hydrogel material 530 is a PEG material. In another embodiment, the hydrogel material 530 is a PLGA material. Various different types of delivery vehicles, such as the delivery vehicles 500, 510, and 520 may be dispersed throughout the hydrogel material 530 to application to a PN allograft. In one embodiment, the hydrogel material 530 is photopolymerizable. In operation, after performing a PN allograft, the hydrogel material having drug loaded delivery vehicles disposed therein is injected locally to the nerve cavity around the PN allograft. The hydrogel material is then photopolymerized to improve the structural integrity of the material within the nerve cavity around the PN allograft.

As described above, small molecules can be retained in and released from hydrogel architectures that are more suited to viably maintain encapsulated materials. By utilizing various combinations of enzymatically and hydrolytically degrading PEG materials, in combination with the PLGA material of the nanoparticles, various ISV agents, Tregs cells, antibodies, and/or MSCs may be released in a time controllable manner via an in situ polymerizable hydrogel material.

PEG hydrogels provide for synthetically modifying the backbone and crosslink architecture, and therefore, the mechanical and diffusive properties of the hydrogel network. The mechanism and kinetics of degradation affect network properties over time as crosslinks break in response to environmental stimuli. Examples of degradation motifs include hydrolysis, thermoresponsive de-gelation, optical cleavage of crosslinks, and enzymatic degradation. Combining the degradation motifs and blending functional PEG copolymers generate hydrogels with complex porosity and mesh size distributions over time and space. Accordingly, any pharmacological compounds loaded within the hydrogel network will be released when crosslink degradation sufficiently opens the network for diffusion.

Enzymatically degradable particles degrade in response to MMPs secreted by the allograft or native axon. Accordingly, successively longer lag times before PLGA particles are exposed and begin to degrade can be achieved. Modulating the particle size distribution provides smooth and tunable release profiles. Release profiles of ISV agents, Tregs cells, antibodies, MSCs, and cells are a function of particle structure, size, and encapsulating phase composition. For example, the encapsulation of ISV agents loaded PLGA nanoparticles within enzymatically degradable PEG hydrogel particles can be protected by encapsulating the PLGA nanoparticles within a hydrolytically degradable PEG-PLA hydrogel. The PEG-PLA matrix is believed prevent the diffusion of MMPs to the core, thus preventing erosion of the enzymatically degradable encapsulant until the programmed degradation of the PEG-PLA shell is complete.

The injectable delivery system further includes microfluidically-generated microscale particles suspended within a continuous PEG-based hydrogel-forming solution that will be polymerized in situ at the PN allograft site. Immunosuppressive agents or cells may be enclosed within either compartment. Both microscale hydrogel droplets and continuous carrier phase materials are to be crosslinked with a variety of hydrolytically and enzymatically degradable motifs to provide control over the spatial distribution of degradation rate of ISV agents.

In-situ delivery of ISV agents via the hydrogel materials described herein at the time of allotransplantation are contemplated to reduce or eliminate the probability of graft rejection by the host biological system. For example, the the ISV agents may reduce or eliminate the probability of graft rejections for a period of time sufficient to enable replacement of donor cells by host cells or for a period of time sufficient for complete regeneration at the graft site. It is also contemplated that localized supplemental injections of the ISV agents may be utilized at or near the allograft site to further suppress the potential for graft rejection post-transplantation.

For example, the ISV agents may be injected, with or without a biomaterial carrier (e.g. hydrogel materials) into tissue which surrounds the graft site prior to placement of the graft into the host. In one example, muscle or connective tissue surrounding a nerve graft site may be injected with ISV agents/cells to locally increase immunosuppressive activity within the vicinity of the graft. In one embodiment, ISV agents/cells are mixed with a biodegradable or biocompatible carrier materials, such as the hydrogel materials described supra, to localize the ISV agents at the time of placement to serve as a reservoir from which the ISV agents and can be locally released over a period of time. In further embodiments, ISV agents injected into tissue surrounding the graft site with a biomaterial carrier may be polymerized to improve retention of the ISV agents about the graft site.

### Graft Microinjection

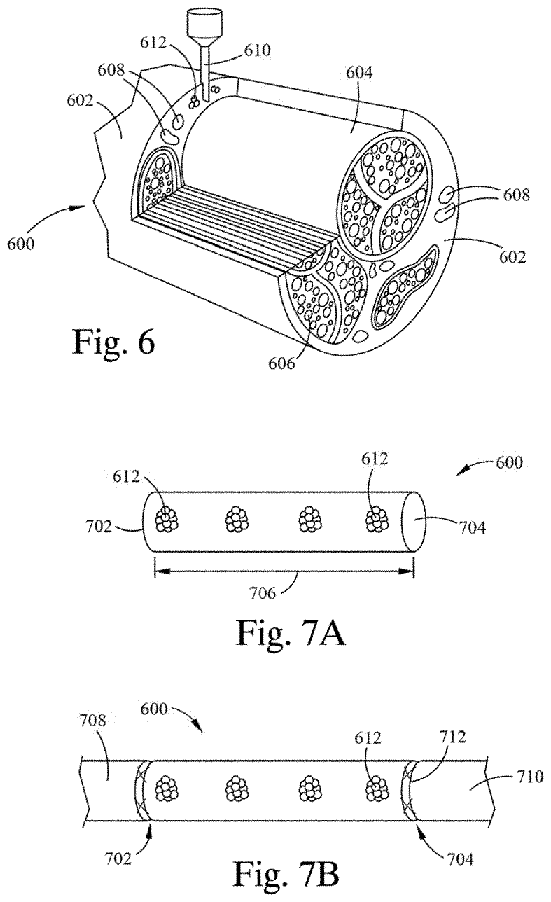

FIG. 6 illustrates a perspective sectional view of a nerve graft 600 injected with immunosuppressive agents 612 according to embodiments described herein. The nerve graft 600 is representative of a peripheral nerve graft and may be an allogeneic graft, autologous graft, or a xenogeneic graft. For example, an allogeneic graft for implantation into a human may be harvested from a different human donor. An autologous graft is one where the graft donor is the same individual as the recipient. For example, an individual's sural nerve may be utilized as a graft and implanted into a different region of the individual's peripheral nervous system. A xenogeneic graft is one where the donor is a different species from the recipient. The nerve graft 600 includes the epineurium 602 which surrounds fascicles 604. Blood vessels 608 extend through the epineurium 602 or tissue between the epineurium 602 and the fascicles 604. Nerve fibers 606, which include axons and the like, are disposed within the fascicles 604.

In one embodiment, the ISV agents 612 are injected into or directly beneath the epineurium 602. In another embodiment, the ISV agents 612 are injected into tissue or interstitial space further beneath the epineurium 601. ISV agent injection into the nerve graft 600 is performed by an injection device 610, such as a needle, micropipette, or other suitable injection apparatus. In one embodiment, the injection device 610 is a hypodermic needle or the like having an inner diameter (lumen) of between about 2 micrometers and about 500 micrometers. Injection of the ISV agents 612 via the injection device may be assisted with a microscope or other imaging device to assist with depth determination. A micromanipulator (not shown) may also be utilized to facilitate proper placement of the injection device 610 into the nerve graft 600. In one example a microinjection syringe available from World Precision Instruments is utilized as the injection device 610.

In the illustrated embodiment, the ISV agents 612 are injected into the nerve graft 600 in an orientation normal to proximal and distal ends of the nerve graft 600. It is believed that injection of the nerve graft 600 at an orientation normal to the ends of the nerve graft 600 prevents or substantially reduces the probability of axonal tract compression. By avoiding axonal tract compression, it is believed the reinnervation post-implantation may be improved. Alternatively, the ISV agents 612 may be injected into the nerve graft through the proximal and distal ends of the nerve graft 600.

FIG. 7A illustrates a schematic view of the nerve graft 600 with the immunosuppressive agents 612 disposed therein according to embodiments described herein. In one embodiment, the ISV agents 612 are injected into the nerve graft 600 in regions adjacent or immediately adjacent to the proximal end 702 and distal end 704 of the nerve graft 600. By injecting the ISV agents 612 into regions of the nerve graft 600 in close proximity to the ends 702, 704, immunosuppression can be concentrated at regions of the nerve graft 600 where graft rejection is most prevalent post-implantation.

In other embodiments, the ISV agents 612 are injected into the nerve graft 600 at a plurality of regions along a length 706 of the nerve graft 600. For example, additional ISV injections may be performed at regions between the ends 702, 704 of the nerve graft 600. While the length 706 of the nerve graft 600 is typically between about 1 mm and about 40 cm, any number of injection sites may be utilized to distribute the ISV agents 612 throughout the nerve graft 600. In one embodiment, the concentration of ISV agents 612 is greater near the ends 702, 704 of the nerve graft 600 when compared to a region of the nerve graft 600 between the ends 702, 704. The concentration of ISV agents may be determined, at least in part, by the volume of ISV agents and/or carrier material injected. In certain embodiments, a volume of ISV agents and/or carrier material Accordingly, it is contemplated that the number of injection sites and the concentration of ISV agents 612 may be constant or varied along the length 706 of the nerve graft 600.

While the aforementioned embodiments contemplate injection of ISV agents into the nerve graft, it is contemplated that other immunomodulatory materials can be injected instead of or in addition to the ISV agents. For example, materials which promote nerve regeneration and/or reinnervation may be injected into the nerve graft 600. Examples of materials which promote nerve regeneration and/or reinnervation include, but are not limited to stem cells, Schwann cells, astrocytes, and radial glia or the like. Other immunomodulatory materials which promote nerve regeneration and/or reinnervation include proteins and peptides, such an chondroitenase ABC and neurotrophic proteins, such as bran-derived neurotrophic factor (BDNF), nerve growth factor (NGF), fibroblast growth factor (FGF), and neurotrophins, such as neurotrophin-3, neurotrophin-4, glial cell line derived neurotrophic factor, neurturin, artemin, and persephin, among others. It is further contemplated that other immunomodulatory materials, such as antibodies, drugs, or pharmaceuticals may be injected to promote nerve regeneration and/or reinnervation.

FIG. 7B illustrates a schematic view of the nerve graft 600 loaded with the immunosuppressive agents 612 grafted into a host according to embodiments described herein. After injection of the ISV agents 612, the nerve graft 600 is implanted into the host. For example, the proximal end 702 of the nerve graft 600 is connected to a proximal host nerve segment 708 and the distal end 704 of the nerve graft 600 is connected to a distal host nerve segment 710. The nerve graft 600 is implanted and secured to the host nerve segments 708, 710 by sutures 712 or other suitable coupling apparatus or materials.

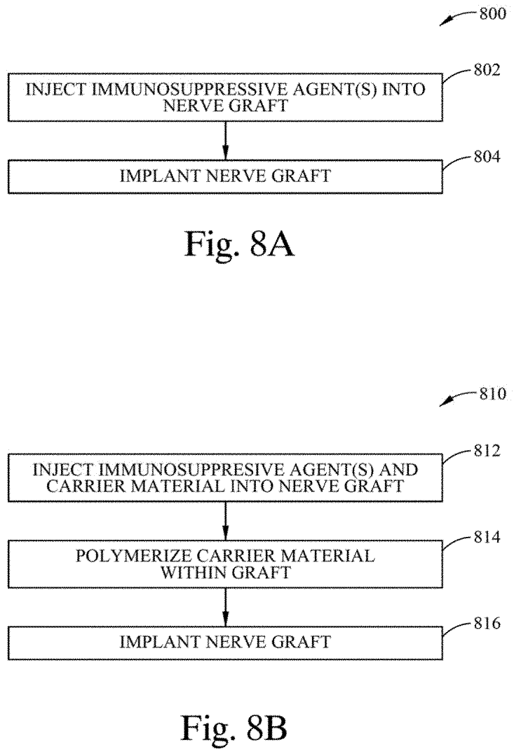

FIG. 8A illustrates operations of a nerve graft ISV agent injection process 800 according to embodiments described herein. At operation 802, ISV agents are injected into a nerve graft. Examples of suitable injection operations are described in detail with regard to FIG. 6 and FIG. 7A. At operation 804, the nerve graft is implanted into a host. Such implantation is described in greater detail with regard to FIG. 7B. By injecting the nerve graft with ISV agents prior to implantation, implantation processes and outcomes may be improved because the efficiency of the implantation process may be improved and the probability of complications associated with implantation may be reduced.

FIG. 8B illustrates operations of a nerve graft ISV agent injection process 810 according to embodiments described herein. At operation 812, ISV agents and a carrier material are injected into a nerve graft. Suitable carrier materials include the hydrogel materials and biomaterial carriers described herein. At operation 814, the carrier material is polymerized within the nerve graft. The carrier material, which includes the ISV agents loaded therein, is polymerized in-situ after the carrier material and ISV agents have been implanted into the nerve graft. In one embodiment, the carrier material is photopolymerized within the nerve graft. UV light, having a wavelength between about 10 nm and about 400 nm, is applied to the nerve graft and the UV light penetrates the graft tissue to facilitate polymerization of the carrier material. In one embodiment, the nerve graft injected with the carrier is exposed to UV light for a duration of between about 10 seconds and about 60 seconds. The UV exposure may be repeated for a similar duration depending upon the intensity of UV light applied to the nerve graft, the material utilized for the carrier, and the degree of carrier material polymerization desired. At operation 816, the nerve graft is implanted into a host.

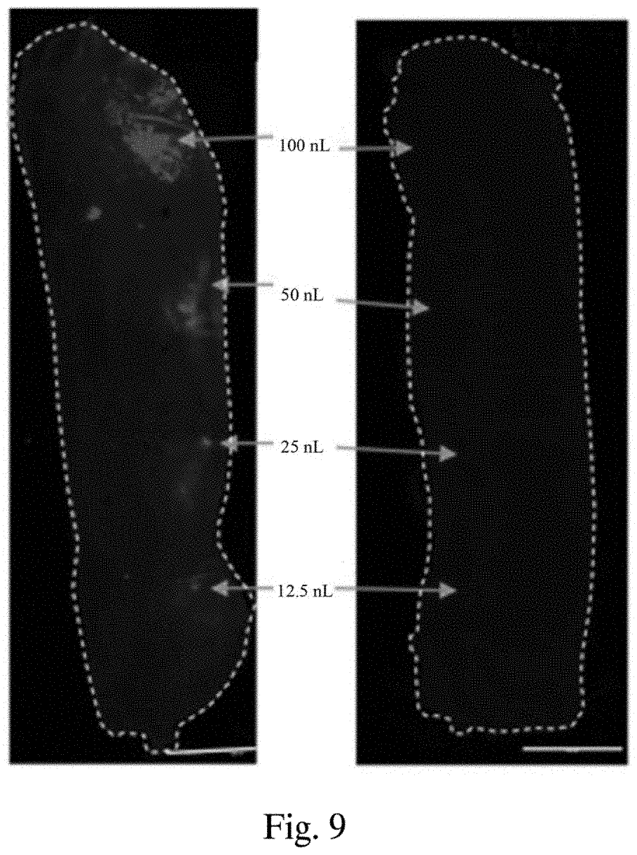

FIG. 9 illustrates images of a nerve graft injected with different volumes of fluorescent beads and a biomaterial carrier according to embodiments described herein. To illustrate the principle of ISV agent injection into a nerve graft, various volumes of red fluorescent beads, 2 micrometer diameter average, were injected into a nerve graft with a carrier solution (left image) and a nerve graft was injected with the carrier solution without the fluorescent beads (right image). Each of the nerve grafts (dotted outline representing the edges of the graft) were injected in four distinct regions with different amounts of fluorescent beads/carrier solution. The left image illustrates increasing fluorescent intensity from the fluorescent beads as the injection volume increases from 12.5 n, 25 nL, 50 nL to 100 nL. The image on the right, which had the same volumes of carrier without fluorescent beads injected, exhibited no fluorescence. The red fluorescent beads are representative of ISV agents and illustrate how ISV agents can be injected into a nerve graft.

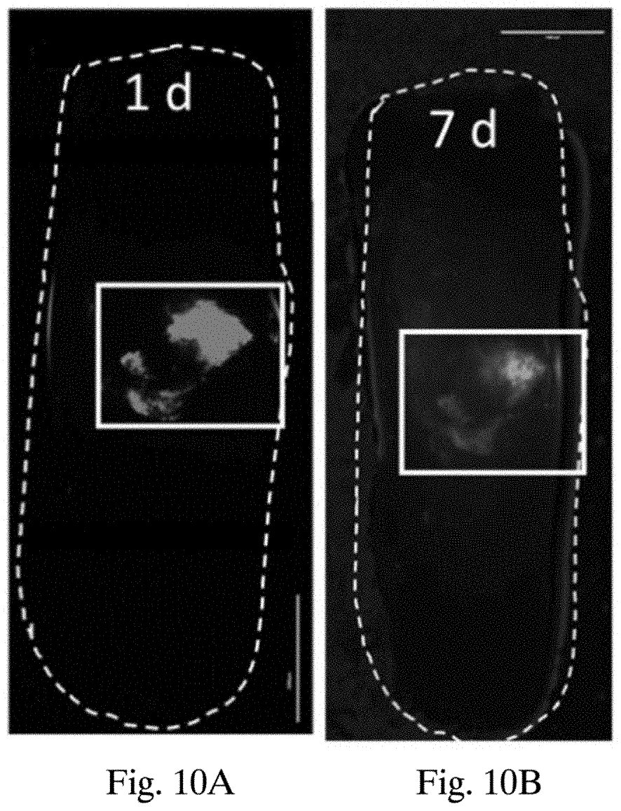

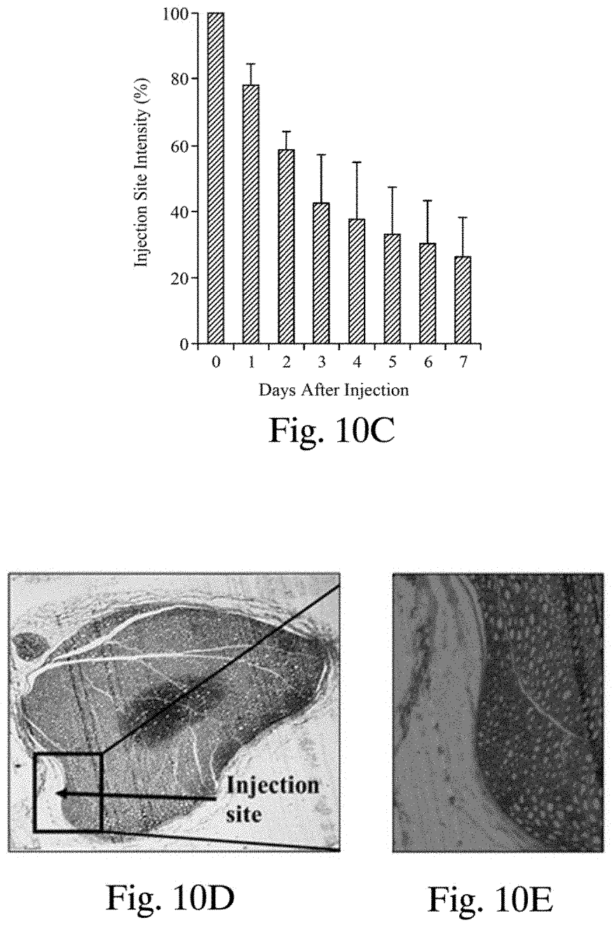

While ISV agents can be injected into a nerve graft without a polymerizable carrier, the ISV agents may exhibit some leakage from the graft at the injection site. FIG. 10A illustrates an image of a nerve graft injected with fluorescent beads within a biomaterial carrier one day after injection according to embodiments described herein. In the illustrated image, green fluorescent beads were injected with a poly(ethylene glycol) norbornene carrier material. After injection of the beads within the carrier material, UV light was applied to the graft to polymerize the carrier within the nerve graft which contained the fluorescent beads. Imaging of the nerve graft revealed bright green fluorescence at the injection site (designated by the white box).

FIG. 10B illustrates an image of a nerve graft injected with fluorescent beads within a biomaterial carrier seven days after injection according to embodiments described herein. The nerve illustrated in FIG. 10A was stored for seven days and imaged each day. The fluorescence illustrated on day seven indicates that the polymerized carrier material kept the fluorescent beads within the nerve graft and substantially reduced or prevented leakage of the fluorescent beads from the injection site.

FIG. 10C is a graph illustrating injection site fluorescent intensity around the injection site for a period of seven days corresponding to the images of FIGS. 10A and 10B according to embodiments described herein. Densitometry of the fluorescent intensity at the injection site was quantified and illustrated that the intensity reduced over time. The intensity reduction indicates that the carrier material was degrading and releasing the fluorescent beads from the carrier material matrix. Thus, the fluorescent intensity at the injection site was gradually lowered over time. The data indicates that ISV agents can be released from a polymerized carrier material within the nerve graft stably over time to provide for sustained localized immunosuppression.

FIG. 10D is an image illustrating a cross section nerve morphometry at the injection site of FIGS. 10A and 10B from toluidine blue staining according to embodiments described herein. FIG. 10E is a close up view of the injection site of the nerve graft illustrated in FIG. 10D. The illustrated morphometrical analysis of the nerve graft cross-section showed that the injection did not significantly disrupt the organization of the nerve. Thus, it is believed that ISV agents and polymerizable carriers may be injected into nerve grafts to provide localized immunosuppression without adversely impacting nerve regeneration and/or reinnervation.

Direct injection of ISV agents directly into the nerve graft provides for sufficient localized immunosuppression to prevent or substantially reduce the probability of graft rejection. For example, the injected ISV agents are believed to provide immunosuppression against the graft by directly contacting and inhibiting immune cells via factors secreted by the injected cells. By injecting the nerve graft directly, immunosuppression is further highly localized to the graft tissue itself and eliminate more regional immunosuppression surrounding the graft. It is also contemplated that injection of ISV agents into tissue surrounding and in the vicinity of the graft may provide for further localized immunosuppression.

In additional to injection of ISV agents into the nerve graft, ISV agents may further be injected into tissue surrounding the nerve graft. For example, ISV agents may be injected into host nerve segments or adjacent musculature or other tissue that is within the vicinity of the nerve graft. Beyond nerve grafts, other tissue is believed to benefit from the methods and materials described herein. For example, a blood vessel can be grafted into a host and the graft site can be surrounded by a polymerizable carrier materials loaded with ISV agents to facilitate localized immunosuppression of the graft. In another example, ISV agents can be injected into the blood vessel graft prior to implantation of the graft.

Graft tissues described herein, including nervous tissue and non-nervous tissue, can be allogeneic, autologous, or xenogeneic. The graft tissue may be freshly harvested, stored, of processed tissue. Grafted tissue and cells may be harvested from the vascular tissue, musculoskeletal tissue, heart tissue, skin tissue, nervous tissue, digestive tissue, organs, ductal tissue, and reproductive tissue. Such grafted tissue may be supplemented with ISV agents or immunomodulatory agents to facilitate immunosuppression or tissue integration within the host.

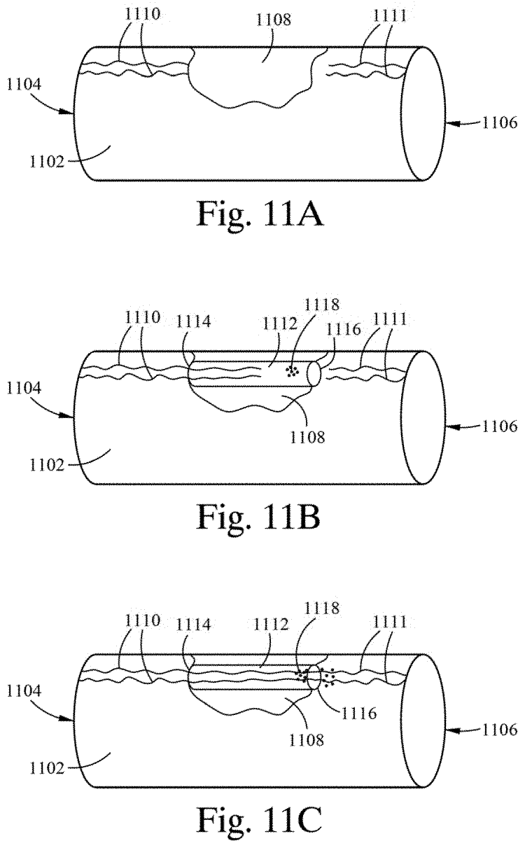

FIG. 11A illustrates a schematic representation of a spinal cord 1102 with a spinal cord injury site 1108 according to embodiments described herein. For purposes of illustration, the spinal cord 1102 includes a proximal end 1104 and a distal end 1106. The injury site 1108 of the spinal cord 1102 is damaged such that proximal CNS axons cannot cross the injury site 1108 and reinnervate with distal CNS axons 1111.

FIG. 11B illustrates a schematic representation of a peripheral nerve graft 1112 into the spinal cord injury site 1108 of FIG. 11A according to embodiments described herein. The nerve graft 1112 is injected with immunomodulatory agents 1118 to facilitate reinnervation of the proximal CNS axons 1110 with the distal CNS axons 111. It is believed that the proximal CNS axons can regenerate and enter a proximal end 1114 of the nerve graft 1112. However, the proximal CNS axons typically cannot exit the nerve graft 1112 to reinnervate with the distal CNS axons 1111.

The immunomodulatory agents 1118 include materials which promote nerve regeneration and/or reinnervation may be injected into the nerve graft 1112. Examples of materials which promote nerve regeneration and/or reinnervation include, but are not limited to stem cells, Schwann cells, astrocytes, and radial glia or the like. Other immunomodulatory materials which promote nerve regeneration and/or reinnervation include proteins and peptides, such an chondroitenase ABC and neurotrophic proteins, such as brain-derived neurotrophic factor (BDNF), nerve growth factor (NGF), fibroblast growth factor (FGF), and neurotrophins, such as neurotrophin-3, neurotrophin-4, glial cell line derived neurotrophic factor, neurturin, artemin, and persephin, among others. It is further contemplated that other immunomodulatory materials, such as drugs or pharmaceuticals may be injected to promote nerve regeneration and/or reinnervation.

The immunomodulatory agents 1118 may be injected into the nerve graft 1112 in a manner similar to operations described with regard to FIGS. 6, 7A, 7B, 8A, and 8B. In one embodiment, the immunomodulatory agents 1118 are alone injected (or in a solution) into the nerve graft 1112. In another embodiment, the immunomodulatory agents 1118 are injected into the nerve graft 1112 with a polymerizable carrier. In this embodiment, the immunomodulatory agents 1118 and the carrier are injected into the nerve graft 112 and polymerized prior to implantation of the nerve graft 1112 into the injury site 1108. Alternatively, the carrier may be polymerized after the nerve graft 1112 is implanted.

In one embodiment, the immunomodulatory agents 1118 are injected into the nerve graft 1112 adjacent to a distal end 1116 of the nerve graft 1112. Because the proximal CNS axons can enter the nerve graft 1112 but cannot typically exit the nerve graft 1112, the immunomodulatory agents 1118 injected into the distal end 1116 of the nerve graft 1112 provide reintegration factors in a region that facilitates axonal growth. In another embodiment, the immunomodulatory agents 1118 are injected into the nerve graft 1112 adjacent to the proximal end 1114 of the nerve graft. In another embodiment, the immunomodulatory agents 1118 are injected into the nerve graft 1112 at regions adjacent to both of the proximal and distal ends 1114, 1116 of the nerve graft 1112. It is contemplated that the immunomodulatory agents 1118 may also be injected into the nerve graft 1112 at regions between the proximal and distal ends 1114, 1116 of the nerve graft 1112.

FIG. 11C illustrates a schematic representation of a peripheral nerve graft 1112 into the spinal cord injury site 1108 of FIG. 11B and reintegration of proximal CNS axons 1110 within the spinal cord 1102 across the injury site 1108 according to embodiments described herein. Because the nerve graft 1112 functions as a bridge or conduit through which the proximal CNS axons 1110 can grow and the injected immunomodulatory agents 1118 provide a favorable environment for nerve reintegration and reinnervation, the proximal CNS axons 1110 can exit the nerve graft 1112 at the distal end 1116 and reinnervate with the distal CSN axons 111. It is contemplated that the immunomodulatory agents 1118 may also chemotax into the surrounding spinal cord tissue near the distal end 1116 of the nerve graft 1112 and create a favorable environment for reinnervation of the proximal CNS axons 1110 with the distal CNS axons 1111.

In summation, methods and material for localized immunosuppression or immunomodulation of grafts are described herein. Various ISV agents may be utilized alone or in combination with one another to facilitate immunosuppression locally to a tissue graft. Materials which provide for delivery of the immunosuppressive agents enable time released and locally controllable delivery which can be utilized to facilitate integration post-transplant without the risks commonly associated with systemic immunosuppression. Immunomodulatory agents which generate environments suitable to promote tissue growth may also be utilized in conjunction with various tissue grafts to facilitate tissue regeneration.

While the foregoing is directed to embodiments of the present disclosure, other and further embodiments of the disclosure may be devised without departing from the basic scope thereof, and the scope thereof is determined by the claims that follow.

What is claimed is:

1. A method of nerve graft preparation, comprising:
injecting immunosuppressive agents into a nerve graft in a first region of the nerve graft adjacent to a proximal end of the nerve graft and a second region of the nerve graft adjacent to a distal end of the nerve graft.

2. The method of claim 1, wherein the immunosuppressive agents are injected into the nerve graft in an orientation normal to the proximal and distal ends of the nerve graft.

3. The method of claim 2, wherein the immunosuppressive agents are injected into the nerve graft below an epineurium of the nerve graft.

4. The method of claim 2, wherein the immunosuppressive agents are injected into an epineurium of the nerve graft.

5. The method of claim 1, wherein the immunosuppressive agents are injected into the nerve graft with an needle having a lumen with a diameter of between about 2 micrometers and about 500 micrometers.

6. The method of claim 1, wherein the immunosuppressive agents are injected into the nerve graft in a volume of between about 12.5 nL and about 100 nL.

7. The method of claim 1, wherein the immunosuppressive agents comprise one or more of a calcineurin inhibitor, aglucocorticoid, and an immunosuppressive antibody.

8. The method of claim 1, wherein the immunosuppressive agents comprise one or both of Tregs cells and mesenchymal stromal cells.

9. The method of claim 1, further comprising:
injecting the immunosuppressive agents into the nerve graft with a polymerizable carrier material.

10. The method of claim 9, wherein the carrier material is a photopolymerizable carrier material.

11. The method of claim 9, wherein the carrier material comprises one or more of extracellular matrix, collagen, alginate, agarose, peptide-based hydrogels, hyaluronic acid, hyaluronan, keratin, fibronectin, dermatan, poly(sialic acid), fibrin, chitosan, chitin, cholic acid, dextran, dextrin, cellulose, gelatin, gelatinous protein mixtures, laminin, perlecan, aggrecan, glycosaminoglycans, heparin, cholesterol, chondroitin sulfate, polycholesterol, hydroxyapatite, silk, bisphosphonates, tricalcium phosphate, sacran, decellularized extracellular matrix, xanthum gum, starch, pectin, amylopectin, amylose, elastin, chemically modified derivatives thereof, and combinations and mixtures thereof.

10. The method of claim 9, wherein the carrier material comprises one or more of poly(glutamic acid), poly(propylene fumarate), poly(N-hydroxyethyl)-DL-aspartamide, propylene glycol, poly(ethylene oxide), poly(propylene oxide), poly(vinyl alcohol), poly(acrylic acid), poly(hedral oligosilsesquioxane), poly(methacrylic acid), poly(vinyl-pyrrolidone), polyacrylamide, poly(isopropylacrylamide), polyphosphazene, peptides, polyaldehyde, tyrosine-derived polycarbonates, methacrylate, polymethacrylate, N-isopropylacrylamide, acrylamide, acrylate, polyacrylate, poly(lactic acid), poly(glycolic acid), poly(lactide-co-glycolide), poly(3,4-ethylenedioxythiophene), graphene oxide, poly(graphene oxide), polycaprolactone, sodium dodecyl sulfate, vinyl phosphonic acid, poly-dimethylsiloxane, titanium, bioactive glass, carbon nanotubes, silicone, silica, thiolene materials, chemically modified derivatives thereof, and combinations and mixtures thereof.

11. The method of claim 9, wherein the carrier material comprises a hydrogel material comprising three distinct materials arranged as triblock copolymers selected from the group consisting of poly(glutamic acid), poly(propylene fumarate), poly(N-hydroxyethyl)-DL-aspartamide, propylene glycol, poly(ethylene oxide), poly(propylene oxide), poly(vinyl alcohol), poly(acrylic acid), poly(hedral oligosilsesquioxane), poly(methacrylic acid), poly(vinyl-pyrrolidone), polyacrylamide, poly(isopropylacrylamide), polyphosphazene, peptides, polyaldehyde, tyrosine-derived polycarbonates, methacrylate, polymethacrylate, N-isopropylacrylamide, acrylamide, acrylate, polyacrylate, poly(lactic acid), poly(glycolic acid), poly(lactide-co-glycolide), poly(3,4-ethylenedioxythiophene), graphene oxide, poly(graphene oxide), polycaprolactone, sodium dodecyl sulfate, vinyl phosphonic acid, poly-dimethylsiloxane, titanium, bioactive glass, carbon nanotubes, silicone, silica, thiolene materials, chemically modified derivatives thereof, and combinations and mixtures thereof.

12. The method of claim 9, further comprising:
polymeric nanoparticles disposed in the carrier material.

13. The method of claim 12, wherein the immunosuppressive agents are disposed within the polymeric nanoparticles.

14. The method of claim 1, wherein the nerve graft is an autologous nerve, an allogeneic nerve, or a xenogeneic nerve.

15. A nerve graft material, comprising:
a nerve graft comprising immunosuppressive agents disposed within the nerve graft below or within the epineurium of the nerve graft, wherein the immunosuppressive agents are disposed within the nerve graft adjacent to a proximal end and a distal end of the nerve graft.

16. The nerve graft material of claim 15, wherein the immunosuppressive agents comprise one or more of a calcineurin inhibitor, a glucocorticoid, and an immunosuppressive antibody.

17. The nerve graft material of claim 15, wherein the immunosuppressive agents comprise one or both of Tregs cells and mesenchymal stromal cells.

18. The nerve graft material of claim 15, further comprising:
a carrier material disposed within the nerve graft adjacent to the proximal end and the distal end of the nerve graft.

19. The nerve graft material of claim 18, wherein the carrier material is a photopolymerizable material.

20. The nerve graft material of claim 18, wherein the carrier material comprises one or more of extracellular matrix, collagen, alginate, agarose, peptide-based hydrogels, hyaluronic acid, hyaluronan, keratin, fibronectin, dermatan, poly(sialic acid), fibrin, chitosan, chitin, cholic acid, dextran, dextrin, cellulose, gelatin, gelatinous protein mixtures, laminin, perlecan, aggrecan, glycosaminoglycans, heparin, cholesterol, chondroitin sulfate, polycholesterol, hydroxyapatite, silk, bisphosphonates, tricalcium phosphate, sacran, decellularized extracellular matrix, xanthum gum, starch, pectin, amylopectin, amylose, elastin, chemically modified derivatives thereof, and combinations and mixtures thereof.

21. The nerve graft material of claim 18, wherein the carrier material comprises one or more of poly(glutamic acid), poly(propylene fumarate), poly(N-hydroxyethyl)-DL-aspartamide, propylene glycol, poly(ethylene oxide), poly(propylene oxide), poly(vinyl alcohol), poly(acrylic acid), poly(hedral oligosilsesquioxane), poly(methacrylic acid), poly(vinyl-pyrrolidone), polyacrylamide, poly(isopropylacrylamide), polyphosphazene, peptides, polyaldehyde, tyrosine-derived polycarbonates, methacrylate, polymethacrylate, N-isopropylacrylamide, acrylamide, acrylate, polyacrylate, poly(lactic acid), poly(glycolic acid), poly(lactide-co-glycolide), poly(3,4-ethylenedioxythiophene), graphene oxide, poly(graphene oxide), polycaprolactone, sodium dodecyl sulfate, vinyl phosphonic acid, poly-dimethylsiloxane, titanium, bioactive glass, carbon nanotubes, silicone, silica, thiolene materials, chemically modified derivatives thereof, and combinations and mixtures thereof.

22. The nerve graft material of claim 18, wherein the carrier material comprises a hydrogel material comprising three distinct materials arranged as triblock copolymers selected from the group consisting of poly(glutamic acid), poly(propylene fumarate), poly(N-hydroxyethyl)-DL-aspartamide, propylene glycol, poly(ethylene oxide), poly(propylene oxide), poly(vinyl alcohol), poly(acrylic acid), poly(hedral oligosilsesquioxane), poly(methacrylic acid), poly(vinyl-pyrrolidone), polyacrylamide, poly(isopropylacrylamide), polyphosphazene, peptides, polyaldehyde, tyrosine-derived polycarbonates, methacrylate, polymethacrylate, N-isopropylacrylamide, acrylamide, acrylate, polyacrylate, poly(lactic acid), poly(glycolic acid), poly(lactide-co-glycolide), poly(3,4-ethylenedioxythiophene), graphene oxide, poly(graphene oxide), polycaprolactone, sodium dodecyl sulfate, vinyl phosphonic acid, poly-dimethylsiloxane, titanium, bioactive glass, carbon nanotubes, silicone, silica, thiolene materials, chemically modified derivatives thereof, and combinations and mixtures thereof.

23. A method of nerve grafting, comprising:
harvesting a nerve graft from a donor;
injecting immunosuppressive agents into proximal and distal regions of the nerve graft; and
implanting the nerve graft into a host.

24. The method of claim 23, wherein the injecting immunosuppressive agents into the nerve graft is performed prior to transplanting the nerve graft into the host.

25. The method of claim 23, wherein the immunosuppressive agents are injected into the nerve graft with a carrier material.

26. The method of claim 25, wherein the carrier material is photopolymerized after injection of the carrier material into the nerve graft and prior to implanting the nerve graft into the host.

27. The method of claim 23, wherein the immunosuppressive agents are injected into the nerve graft in an orientation normal to proximal and distal ends of the nerve graft.

28. The method of claim 27, wherein the immunosuppressive agents are injected into the nerve graft below an epineurium of the nerve graft.

29. The method of claim 27, wherein the immunosuppressive agents are injected into an epineurium of the nerve graft.

30. The method of claim 23, further comprising:
injecting immunosuppressive agents into one or more host tissues surrounding or adjacent to a graft site of the nerve graft.
